(19) United States
(12) Patent Application Publication
(10) Pub. No.: US 2008/0195190 A1
(43) Pub. Date: Aug. 14, 2008

(54) Flexible Intravascular Implant

(75) Inventors: Thiemo Bland, Plankstadt (DE); Markus Lorenz, Karlsruhe (DE); Thilo Wack, Durmersheim (DE); Benn Beagan Miller, Cambridgeshire (GB); Russell Farn, Westwood Heath Coventry (GB); Simon Grover, Oxfordshire (GB); Dominic Webber, Cambridgeshire (GB)

Correspondence Address: PROSKAUER ROSE LLP, 1001 PENNSYLVANIA AVE, N.W.,, SUITE 400 SOUTH, WASHINGTON, DC, 20004, US

(73) Assignee: ANGIOMED GMBH & CO. MEDIZINTECHNIK KG, Karlsruhe (DE)

(21) Appl. No.: 11/572,744

(22) PCT Filed: Aug. 1, 2005
(86) PCT No.: PCT/EP2005/008319

§ 371 Date: Jan. 23, 2008

(30) Foreign Application Priority Data
Jul. 30, 2004 (GB) 0417078.3

(51) Int. Cl. A61F 2/84; A61F 2/82

(52) U.S. Cl. 623/1.11; 623/1.15

(57) ABSTRACT

The stent matrix of a stent incorporates joints that permit stress-free relative movement of first and second structural portions (10, 12) of the matrix as the facing surfaces of the joint between the structural portions slide relative to one another. The joints are formed in the thickness of an annular wall of the stent, and are created by a computer-controlled laser beam cutting procedure.

### FIELD OF TECHNOLOGY

This invention relates to medical implants for bodily lumens which exhibit first and second structural portions subject, in use, to stresses that are relieved by movement of the portions, one relative to the other.

### SUMMARY OF THE INVENTION

When a stent is to be delivered to a stenting site through a tortuous body lumen, at the distal end of a catheter, the flexibility of the stent enables it to undergo various forms of deformations, for example, bending so that its longitudinal axis is no longer straight but curved, twisting around its longitudinal axis so that its ends rotate relative to each other with the longitudinal axis as its axis of rotation, or with compression or extension of the length of the stent along its longitudinal axis as it moves with the bodily tissue in which it is implanted. Clearly, the more force it takes to deform the stent, the more difficult it is to advance the catheter delivery system, including the stent, along the tortuous lumen, and the higher the risk of damage to the walls of the lumen.

Even after deployment within the body, a stent is subject to stresses that can be relieved by flexing. The amount of strain which the stent is called upon to accommodate varies from location to location within the body but flexibility after placement is advantageous in most (if not all) stent applications, and highly advantageous in many applications. Ideally, a “forceless” or “stressless” bending of the stent is required. One wants the stent to bend without such bending imposing forces or stresses in the bodily tissue in contact with the stent. After all, most bodily tissue except bone has capacity for flexing, and stents placed in soft tissue should be able to flex with that tissue whenever called upon to do so. However, one also needs mechanical integrity between one end of the stent and the other, if only to ensure that the stent can be smoothly deployed progressively from one end of the stent to the other, and that all parts of the stent will remain, after deployment, in the correct location and orientation relative to each other.

One way to endow the stent matrix with substantial flexibility is to provide stent struts that are relatively bendable, and link the successive stenting rings that provide radially outward force to hold back the bodily tissue defining the stented lumen. Another way is to rely upon lengthy connectors. Flexibility increases with connector length, so connectors with a pronounced meander form are common. However, strain is not always a good thing. Managing strain throughout the stent matrix is a considerable challenge.

The essence of the present invention is to use a joint, either to avoid strain in a flexible connector, or to reduce such strain. Characteristic of any joint in accordance with the present invention are first and second facing joint surfaces between which there is relative translational movement. The first joint surface is on a first structural portion of the stent matrix and the second joint surface is on a second structural portion of the stent matrix, so that relative movement between the first and second structural portions can be accommodated by the said translational movement without requiring elastic or plastic deformation of any part of the stent. If there is no elastic or plastic deformation, then there is no resultant forces imposed by the stent on the bodily tissue.

The translational movement will be resisted by static then dynamic friction. However, within the designed range of relative movement between the joint surfaces, such movement will not be opposed by a stress which, in the prior art, is increasing with the displacement between the first and second structural portions away from an original or at rest disposition of the first and second structural portions relative to each other since the first structural portion and the second structural portion are not subject to elastic or plastic deformations but slide face-to-face relative to each other.

It is conventional to create a stent matrix of stenting rings and “flexible” connection portions by laser cutting it out of tubular feedstock, typically of stainless steel or a nickel-titanium shape memory alloy (such as NITINOL®). It is conventional to cut the flexible connectors in the same operation as the formation of the stent matrix. In preferred embodiments of the present invention, the laser is used to create the aforementioned joint surfaces from within a tubular feedstock workpiece, that is, taking both the first and second structural portions from a single workpiece (in a somewhat similar but not quite the same way that a jig saw puzzle is created from a single sheet of plywood). Nevertheless, the present invention is not restricted to implants cut from a single workpiece. Implants can be assembled from portions deriving from separate workpieces and may be of different materials. The structural portions each side of the joint interface could be assembled in “snap-fit” manner, or bonded together in ways known to persons skilled in such micro-assembly techniques.

It is conventional to mount the workpiece relative to the cutting beam of the laser in a jig that moves the workpiece (under computer control) in rotation about the long axis of the workpiece and in translation along the long axis, with the beam at all times on the line that intersects with said long axis. In this way, all laser cuts lie on a plane that passes through the long axis and are therefore perpendicular to the tangent of the outside tubular surface of the workpiece where the beam penetrates through it.

In consequence, surfaces facing each other across a line of a laser cut are free to slide face-to-face relative to each other radially in and out with reference to the said long axis. However, if one were to move the workpiece relative to the laser so the laser beam no longer passes through the long axis of the tubular workpiece, the potential for face-to-face sliding would exist in some other plane, not radial to the long axis. Going a step further, one can envisage a joint in which the joint surfaces have been laser cut in more than one relative orientation, deliberately to set up a steric hindrance to face-to-face sliding, in every direction except the one which is desired of the joint being created. In this way, computer control of the movement of the workpiece relative to the laser beam can create not only the stent matrix but also a plurality of joints between portions of the stent matrix which should be able to move, in use, relative to each other to relieve stresses within the stent matrix, yet also serve to maintain the mechanical integrity of the stent matrix, end-to-end.

The types of joints that are best suited to the present application are not necessarily the ones that one would suppose are simplest and most apt. Rather, they will be the ones that can deliver acceptable performance yet are compatible with the beam-cutting techniques used to create a stent matrix from tube feedstock. For example, to replace a meander-form connector strut one thinks intuitively of a simple hinge joint. Yet it is not immediately apparent how one is to create such a joint by manipulating a tubular workpiece under the beam of a laser cutter. In any case, a simple hinge joint brings little in the way of relative movement between first and second structural portions of a stent matrix that is useful in relieving the stresses in the matrix that arise in actual use of the stent.

In one of many embodiments of the present invention, there is a sliding joint within the wall thickness of the tube stock from which the stent is formed. The present inventor has realised how to include a sliding joint in such a stent matrix made from tube stock.

Another embodiment of the present invention is manifested in a method of making a flexible stent from tube stock, which is characterized by forming a sliding joint within the strut matrix. Such a joint can be made by cutting joint lines through the wall thickness of the tube stock, said joint lines including portions that do not project through the longitudinal central tube axis of the tube stock. The use of such “off-axis” cuts allows steric hindrance between the two tube stock portions, one each side of the sliding surfaces of the joint, to frustrate any tendency of the portions to separate from each other along the joint line.

Besides movements of the tube stock portions in a movement parallel to the longitudinal axis, the use of such slide joints also allows the rotation of the tube stock portion about a short (radial) axis perpendicular to the longitudinal axis of the tube stock. This is possible due to some play between the first node and second node (perhaps due to manufacturing tolerances and the gap produced by the laser) that allows for some hinging movement about the longitudinal axis of the sliding joint. Thus, an arrangement of sliding joints in diametrically opposite pairs, with each structural stent ring of a stenting tube having an axial length between first and second ends of the ring, and with each such ring end jointed by a pair of sliding joints to an adjacent end of the next adjacent stent ring, with one such joint at each end of a diameter to the stent lumen. The defining diameter of the pair of joints at one end of each stent ring is displaced by, say, 90° from the defining diameter of the pair of joints at the other end of the same stenting ring, so as to give a stent made up of a string of such rings the flexibility to bend in all directions away from a straight line on the long central axis of the lumen of the stent.

Such joints could be formed with a frusto-conical joint surface, female on one side of the joint to receive the male frusto-cone of the joint component on the other side of the joint line.

The natural springiness of the material of the tube feedstock tends to retain the male frusto-conical portions within the receiving female frusto-conical seatings of the joints of the joint pair, but this can be supplemented, as desirable or necessary, for example, by capping the base end of the female frusto-conical joint seating surface to prevent the male frusto-cone exiting radially outwardly past the base. One way to cap the base is to employ a brace or bridge piece that spans the male portion and is fixed to the female piece, such as by welding.

A disadvantage of a joint construction with steric hindrance is the need to tilt the workpiece relative to the laser, when cutting the joint surfaces, to orientations in which the laser is not at 90° to the long axis and/or to the short axis of the stent lumen. Even if there is power enough in the CAM software, and movement enough in the jig that presents the workpiece to the laser, there is still the problem that the large dimensions of both the laser system and the tubular workpiece (typically 3 m long) makes it difficult to have movement in more than two degrees of freedom during cutting. However, this can be overcome by, for example, taking a shorter workpiece and mounting it in a jig with enough degrees of freedom of movement relative to the cutting beam to allow three degrees of freedom.

### BRIEF DESCRIPTION OF THE DRAWINGS

For a better understanding of the present invention, and to show more clearly how the same may be carried into effect, reference will now be made, by way of example, to the accommodating drawings, in which:

FIG. 1 is a view from above, of a sliding joint portion in a flexible stent in accordance with the present invention;

FIG. 2 is a section through tube stock, transverse to the long axis of the tube, showing cut-lines;

FIG. 3 is a section through the line iii-iii in FIG. 1; and

FIG. 4 is a section through line iv-iv of FIG. 1.

FIG. 5 is an isometric view of stenting rings interspersed with sliding joints;

FIG. 6 is a view from above, of a sliding joint portion with a reduced thickness portion in accordance with the present invention;

FIG. 7 is a section through line vii-vii of FIG. 6;

FIGS. 8 and 9 are each a section through line ix-ix of FIG. 6;

FIGS. 10A-10B are respective isometric views of two variations of a three-part stent joint in accordance with the present invention;

FIG. 11 shows a view of a three-part stent joint in accordance with the present invention where the central section of the joint are connected to each other.

FIGS. 12 to 14 is a view from above of a rotating joint portion in accordance with the present invention;

FIGS. 15A-15B is a view from above of a sliding joint portion having ramps in accordance with the present invention;

FIGS. 16A-16B is a view of a sliding joint portion having a brace in accordance with the present invention; and

FIGS. 17 to 21 illustrates the various method steps for manufacturing a braced sliding joint portion in accordance with the present invention.

### DETAILED DESCRIPTION

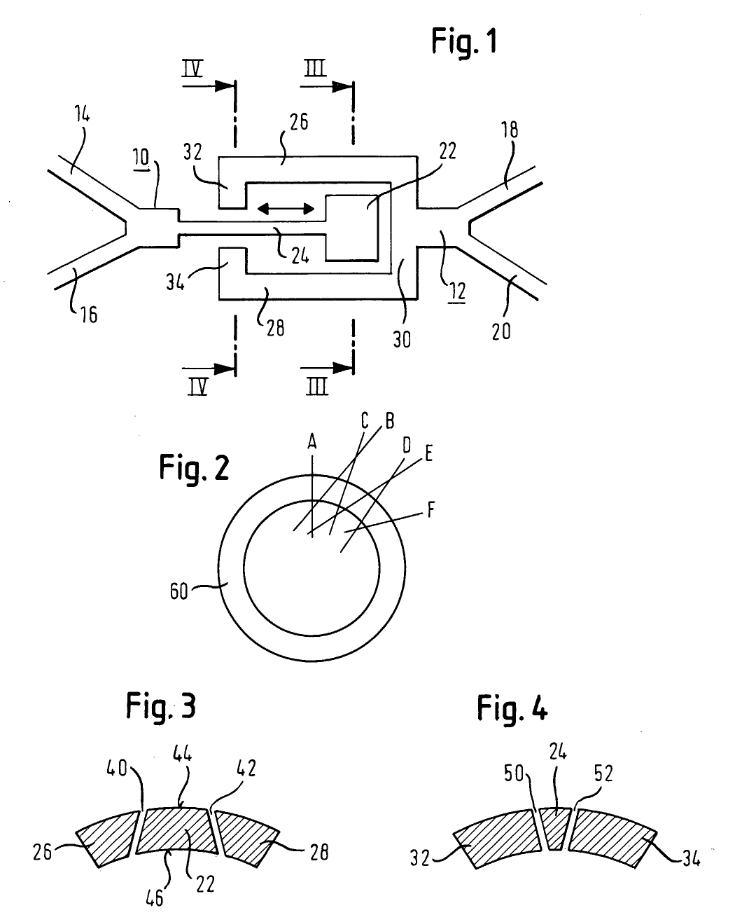

FIG. 1 is a view from above of a sliding joint in a flexible stent in accordance with one exemplary embodiment of the present invention. The joint is in a bridge between a first node 10 and a second node 12 of the stent matrix, the node 10 being between stent matrix struts 14 and 16 and the node 12 between struts 18 and 20. The bridge between the nodes 10 and 12 looks like a piston cylinder arrangement, with a piston head 22 on a piston rod 24 which forms a rigid connection between the node 10 and the piston head 22. The piston head 22 slides a pair of sliding surfaces defined by a “cylinder” rigidly mounted to the node 12. In effect, the cylinder is a pair of rails 26, 28 joined to the node 12 by a back span 30. At the other end of the rails 26, 28 from the back span 30, there are respective opposing clamping portions 32 and 34 which bracket the piston rod 24, such that the piston rod 24 slides on the end surfaces of the clamping portions 32 and 34. Drawing FIGS. 3 and 4 reveal how the facing surfaces slide on each other.

Turning first to FIG. 3, laser cut-lines 40 and 42 are shown through the wall thickness of the tube stock, on a line which does not project through the long axis of the tube stock. Rather, the two cut lines 40 and 42, instead of converging on the long axis of the tube stock, instead diverge. This has the consequence that the abluminal surface 44 of the piston head 22 occupies less surface area than the luminal surface 46 of the piston head. This has the consequence that the piston head 22 cannot move radially outwardly relative to the confining rail struts 26 and 28 of the tube. The piston head 22 could, however, move radially inwardly relative to the rails 26 and 28.

Turning now to FIG. 4, here we see laser cut-lines 50 and 52 converging on a point within the lumen of the tube stock which is between the piston rod 24 and the long axis of the tube stock. In consequence, the piston rod 24 is wedge-shaped in cross-section and can, if isolated from the piston head 22, readily move radially outwardly relative to the clamping portions 32 and 34 of the tube stock either side of it.

It will be appreciated that the rigid connection of both the piston head 22 and piston-rod 24 to the node 10 and stenting ring including struts 14 and 16 inhibits any radial separation of movement of the piston rod 24 and piston head 22. They can move radially with respect to the node 12, only together with both the nodes 10 and 12. The steric hindrance evident from FIGS. 3 and 4 frustrates any radial movement of one of the nodes 10, 12, relative to each other unless the other of the nodes moves with it. More specifically, the cut lines 40, 42 on the sides of the piston head 22 resist any radially outward movement of the node 10 whereas the sliding movement of the piston rod 24 within its clamping portions 32 and 34 restrains any radially inward movement of the node 10 relative to the node 12.

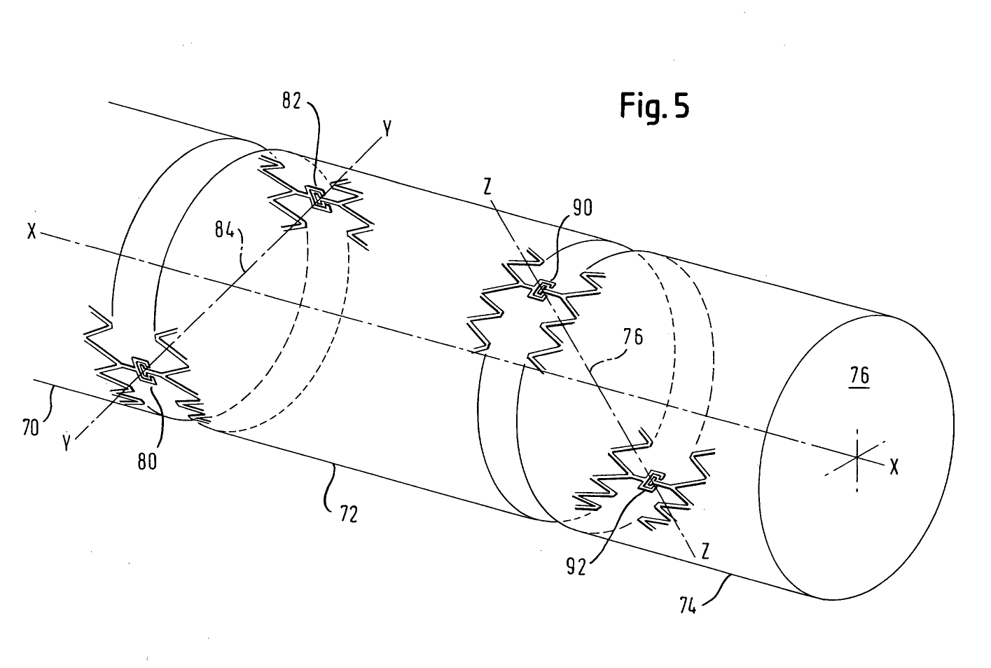

Yet relative translational movement of the nodes 10 and 12 along the long axis of the tube stock is permitted, by sliding of the piston rod and piston head relative to the confining portions of the tube stock 26, 28, 32 and 34, surrounding the piston head and rod. Indeed, this sliding movement is more or less forceless, i.e. not resisted. Thus, a stent matrix including a plurality of sliding joints such as shown in FIG. 1, judiciously placed around the circumference and along the length of the stent matrix, for example as shown in FIG. 5, will provide all the flexibility that is needed for delivery along a tortuous bodily lumen, and for accommodation of bodily movements of the surrounding tissue after placement in the body.

Furthermore, the sliding joint that delivers such flexibility is not provided at the expense of useful cross-sectional area of stent for resisting ingress of bodily tissue into the lumen defined by the stent. On the contrary, the relatively large cross-sectional area of the sliding joint, in the envelope of the stent strut matrix, delivers an enhanced technical effect in keeping open and unobstructed the stented lumen at locations along its length that are in between adjacent stenting rings.

FIG. 2 is provided for the sake of completeness, to show a sequence of lines A to F denoting successive passes of a cutting laser beam through the wall thickness of tube stock 60.

It will be appreciated that the surfaces of the tube stock which have been exposed by laser cutting are liable to be coated with an oxide layer and that oxide layers inhibit electrical continuity and conductivity. Accordingly, a sliding joint in accordance with the present contribution to the art is believed to deliver not only enhanced flexibility but also inhibition of eddy currents flowing within the metallic strut matrix and this is liable to be significantly advantageous in an MRI environment, as explained in Applicant's International Patent Application Publication WO 03/075797, which is hereby incorporated by reference in its entirety into this application.

As explained above, sliding joints are not the only type of joint here contemplated. FIG. 5 is a schematic isometric view of a portion of a stent cut from tubular feedstock that features stenting rings 70, 72, 74 spaced successively at regular intervals along the long axis X-X of the tubular feedstock. Conventionally, each such stenting ring feature a pattern of zig-zag or diamond cell struts going all around the circumference of the lumen 76, with additional struts joining the rings to those on each side that are adjacent. These struts may be relatively long, or a meander-form to give the stent matrix some flexibility to bend out of a straight line corresponding to the long axis X-X.

One could also provide a bending facility, force-free, without imposing any strain on any part of the stent matrix by using joints, for example the sliding joints illustrated in FIG. 1, with facing surfaces that slide over each other to deliver a hinging action. In other words, the portions of the stent either side of the joint rotate relative to one another as opposed to moving in translation relative to each other. Consider FIG. 5 and the consequence of hinging ring 72 downwardly on the page of the drawing about hinge axis Y-Y, and/or rotating ring 74 upwardly about the same hinge axis Y-Y. By placing a sliding joint with its direction of extension and compression oblique to the longitudinal axis of the stent, preferably at 45 to the longitudinal axis, a plurality of such obliquely individual joints delivers a twisting facility, force-free, without imposing any strain on any part of the stent matrix such that one end of the stent can enjoy a range of rotation about the longitudinal axis of the stent, relative to the other end of the stent. Finite element analysis has revealed that such a facility for twisting movements is likely to enhance stent performance in real application.

Between rings 70 and 72 are first and second joints 80 and 82 at opposite ends of the diameter 84 corresponding to hinge axis Y-Y. Each joint has a frusto-conical male joint element received within the flanks of a female joint element with complementary frusto-conical joint surfaces. Relative rotation of stenting rings 70, 72 about the diameter 84 can be accommodated by sliding movement of the frusto-conical joint surfaces of each joint 80, 82 along the axis X-X.

The joints 90, 92 between rings 72 and 74 are at opposite ends of a diameter 76 corresponding to hinge axis Z-Z, which is displaced 90° in orientation on the long axis X-X relative to axis Y-Y, so as not to allow rings 72, 74 to rotate relative to each other than up and down in an orthogonal direction, that is, into and out of the plane of the paper. Thus, stent bending, with rotational movement of the adjacent stenting rings of the stent, relative to each other, in whatever orientation is needed, can be accommodated by using joints located between successive rings at different radial locations along the length of the stent.

An important benefit of the joints of the various exemplary embodiments that characterize the contribution to the art is that they have an inherent capacity to reduce electrical conductivity within the stent matrix. This is enhanced by the tendency of the laser cutting beam to oxidise the feedstock material and leave the joint surfaces to a greater or lesser extent oxidised (with oxide layers being generally far less electrically conductive than the parent metal). Putting breaks in the end-to-end conductivity of a metal stent matrix can be effective to prevent its functioning as a Faraday cage. This effect is important, for example, when one wishes to use MRI techniques to image the lumen defined by the stent.

Although the above description describes cutting with a laser it will be apparent that other cutting techniques are possible, such as by jets of energy (electron beam for example) or jets of fluid (water for example) as well as other cutting techniques such as chemical or electrical etching techniques. Whereas the advantages of the subject-matter disclosed in the present application are evident most readily in metal stents, they are also available in implants other than stents (filters, for example) and materials other than metal (shape memory polymers, for example). In the instant application, the expression “jet cutter” is used as a generic for all the above-noted cutting devices.

Indeed, the ability of the invention to relieve stresses in the stent matrix by movement within the joints will be the increasingly valuable with decline in the ability of the stent matrix to tolerate applied stresses. Whereas springy metals can endure more or less permanently levels of applied stress below the elastic limit (subject to fatigue failure considerations) polymers may show unwanted time-dependent plastic deformation which might be avoidable with the use of the joints of the present invention to replace flexible links and bendy connectors.

Self-expanding stents of nickel-titanium shape memory material are inherently very flexible. Stainless steel balloon-expandable stents are often significantly less able to tolerate large strains. Accordingly, the present invention is believed to be particularly useful and advantageous in the field of stents that are to undergo plastic deformation upon an expansion of diameter by an external deployment agent, such as, for example, as by balloon expansion.

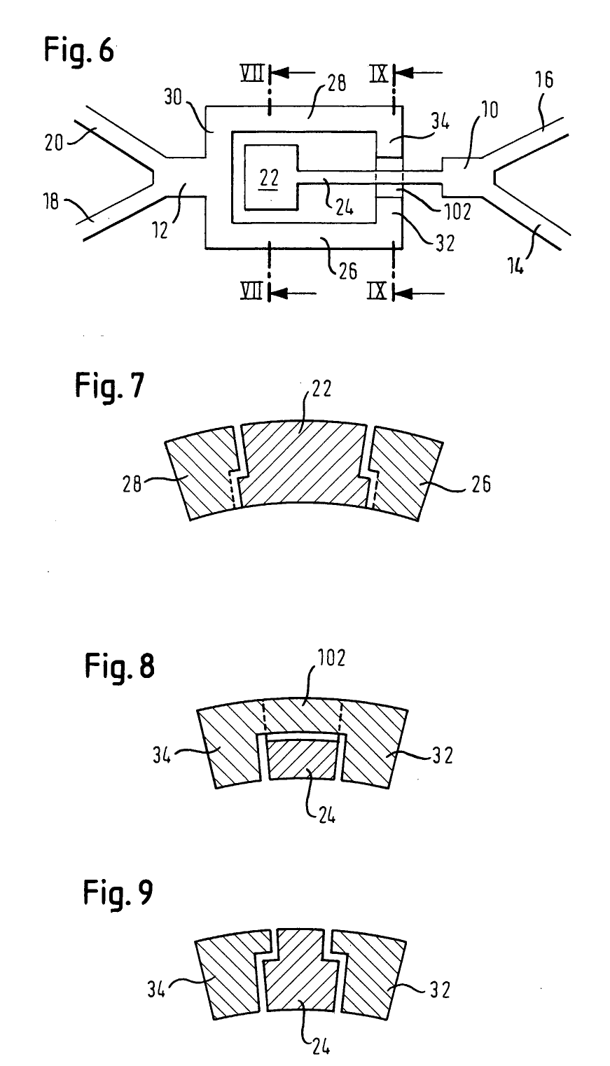

FIG. 6 corresponds to FIG. 1 with the exception that the piston rod 24 is shown passing over a reduced thickness portion 102 spanning the gap between clamping portions 32, 34. FIG. 8 shows a section transverse to the piston rod 24 which reveals this more clearly. In another way to “lock” the piston 22, 24 and cylinder 26, 28 together against relative radial movement a stepped joint interface surface can be created such as is shown in the examples of FIG. 7, which is a section through the line VII-VII in FIG. 6, and in FIG. 9 which is a section through line IX-IX in FIG. 6. Obviously, the direction of the step can be reversed, as needed, to prevent the middle element in each of FIGS. 7 and 9 from moving radially inwardly relative to the portions on either side of it, instead of preventing movement towards the top of the page as shown in the sheet of drawing figures.

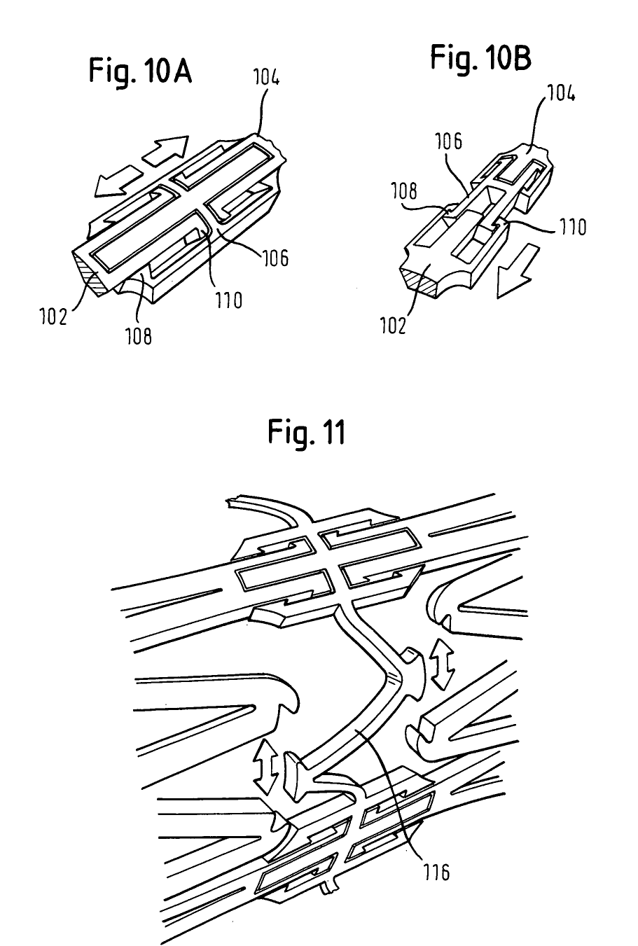

FIG. 10A shows one exemplary embodiment of a three-part sliding joint. FIG. 10B show another exemplary three-part sliding joint for a stent in accordance with the present invention. The illustrated three-part sliding joint has generally three parts: (1) A first structural portion 102, (2) a second structural portion 104, and a center joint part 106. The center joint part could also be a multi-stage element. (Compare the technical field of hydraulic piston cylinder actuators, in particular with multi-stage actuators with each stage being accommodated within the dimension of the stage below.) FIG. 10A illustrates the three-part sliding joint in the non-extended position while FIG. 10B illustrates the three-part sliding joint where one side of the three-part sliding joint is in an extended position and other side is in a non-extended position. One distinction between the embodiment of FIG. 10A and FIG. 10B is that the center joint part 106 can be configured with variations at the detent re-entrant surface portions of the tips 108 of the center joint part 106. For example, in FIG. 10A, the four tips of the center joint 106 are configured with their re-entrant portions to face towards each other whereas, in FIG. 10B, the four detent tips of the center joint 106 are configured to face away from each other. The orientations of the tips of the center joint 106 complement the orientation of the slidable structural portions 102 and 104, and the placement of their detent tips 110, as shown in FIGS. 10A and 10B.

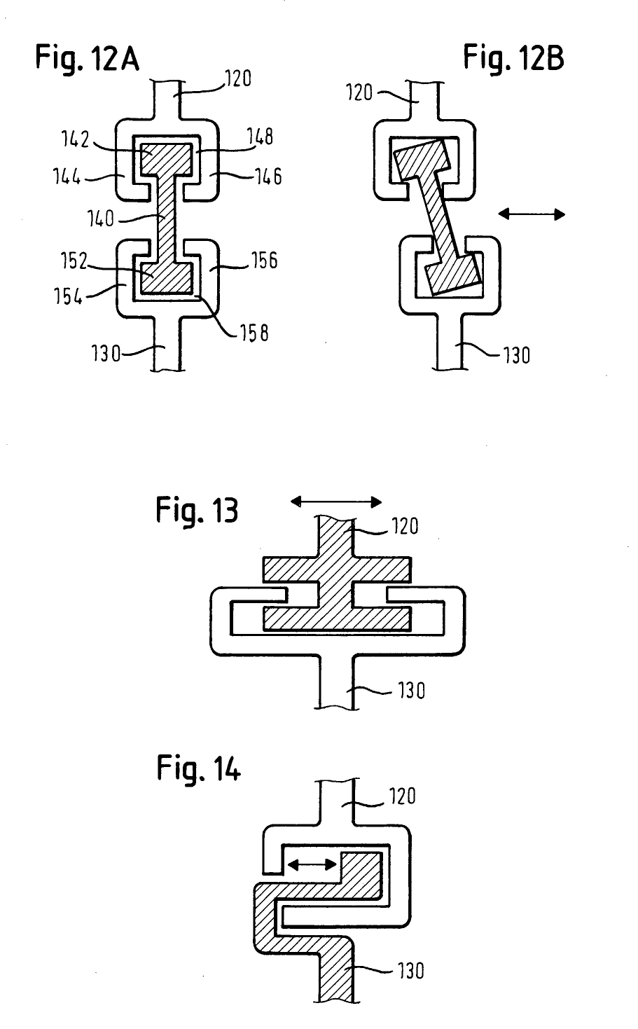

An issue with all joints is that in spite of all reasonable precaution and care in designing and manufacturing such joints there is always the possibility that a center part, for example the rod 140 in the embodiment shown in FIGS. 12A and 12B, can be detached from the joint, and therefore detached from the stent. In the embodiment shown in FIG. 11, a tie element 116 is attached to adjacent center joint parts so that a complete ring around the stent annulus is created by the endless loop formed by four spaced tie elements between four spaced joint center parts. In this way, individual joint center parts cannot inadvertently slip away from the stent and become a detached hazard in the body as the stent or medical implant device is deployed, as shown in FIG. 11.

FIGS. 12A and 12B show an exemplary embodiment of a rotating joint in a flexible stent in accordance with the present invention, which has the capability to provide a rotational movement about the longitudinal axis of the stent between a first structural portion 120 of the stent matrix and a second structural portion 130, thereby allowing a rotational movement between the stenting rings. A double-headed rod 140 connects the two structural potions, with one of its heads 142 embraced by flanks 144, 146 of a receiving cavity 148. The head 152 at the other end of the rod 140 is received in a like cavity 158 defined by flanks 154, 156 cut in the structural portion 130.

Not shown in FIG. 12 is a suitable means to prevent each piston head 142, 152 from moving radially outwardly with respect to the surrounding flank surfaces and the long axis of the stent tube. Such means can be, for example, a bridge extending over the rod 140 like the bridge 102 of FIG. 8. Otherwise, a cover can be provided over the whole of each cavity 148, 158 radially outside the respective flanks, to prevent the rod head 142 or 152 escaping the cavity by moving radially outwardly relative to it. Alternatively a steric hindrance, as similarly illustrated in FIGS. 3-4 could be used to prevent the rod head 142 or 152 escaping the cavity.

Depending on the design parameters and free play between the elements (e.g., due to manufacturing tolerances and the gap produced by the laser), the rotating joint illustrated in FIGS. 12A and 12B is also able to slide in a direction along the longitudinal axis and hinge about the longitudinal axis of the joint, and therefore, could be used in lieu of the sliding joints in the stent or stenting rings illustrated in FIG. 5.

By contrast, the joint structures shown in FIGS. 13 and 14 have no capacity for any substantial rotational hinging movement but only relative translational movement. In overall appearance they are not unlike the meander patterns of known stent designs but, in contrast to the known stent meander patterns, they offer the prospect of a limited amount of movement without requiring elastic or plastic stress within the stent matrix. If enough such joints are provided, enough stent flexibility can be delivered to meet design objectives. With a computer-controlled jig for laser cutting a stent matrix, and enough degrees of freedom to move the workpiece relative to the cutting jet or beam joint configurations such as are described herein can complement, or even fully supplant, the flexible connector struts of conventional stent matrix designs.

One issue in designing joints is to avoid failure at an extreme position of the range of movement of the joint such as the most extended position of a sliding joint, in particular when a force is still applied at the extended position. The present application discloses three possible approaches for resolving or reducing the risk of failure. Furthermore, the present application also discloses one approach to minimize the damage such a failure could cause.

In FIG. 8, the use of a reduced thickness portion 102 spanning the gap between clamping portions 32 and 34 is shown. The reduced thickness portion 102, in effect, acts like a brace, preventing the clamping portions 32 and 34 from moving outwards and releasing the piston 22 (in addition of also preventing radial movements of the piston 22 relative to the clamping portions 32 and 34).

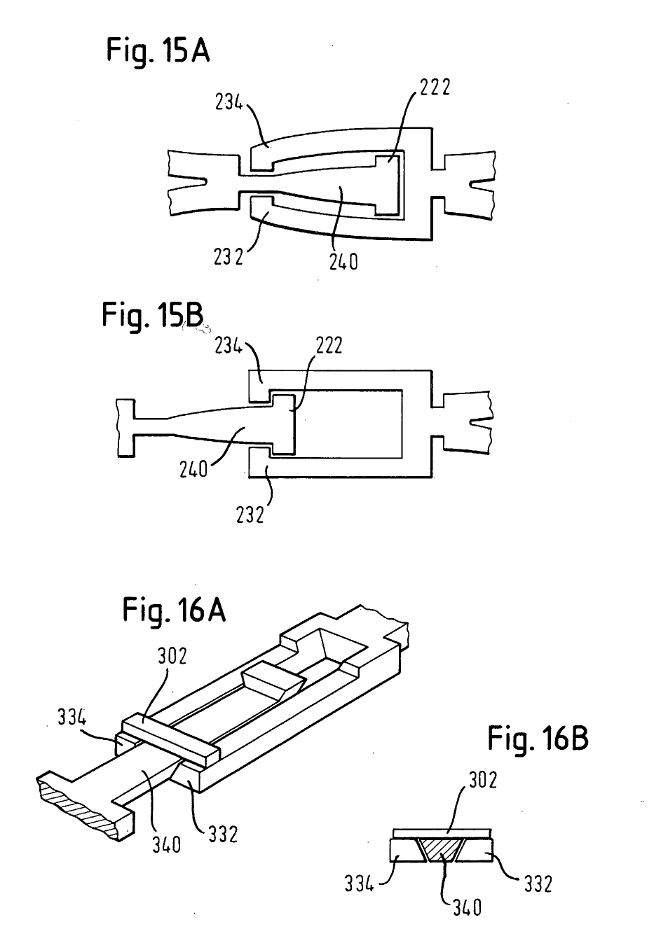

FIGS. 15A-15B shows another approach for preventing the clamping portions from moving outwards by pre-stressing the clamping portions 232 and 234. In FIG. 15A the joint is in its non-extended state, and the clamping portions 232 and 234 are inclined inwards and towards each other in order that the clamping portions are elastically forced apart when the joint is extended and thereby elastically stressed. In FIG. 15B the joint is in its extended state, and the stressed clamping portions 232 and 234 are actively gripping the rod 240. The inwardly inclined clamping portions 232 and 234 are forced outward by the rod 240 that has a width that is increasing as it approaches the head 222 at the rod 240. The inward inclination can be created by bending the parallel portions elastically inwardly, or by jet-cutting the clamping portions with the inwardly-curved shape that will be stressed when the joint in brought to its full extended position.

FIGS. 16A-16B show another approach of preventing the clamping portions 332, 334 from moving outwards by using a brace 302 welded onto the clamping portions 332, 334. The brace 302 also prevents the rod 340 from disengaging from the clamping portions. The brace could be fashioned from tantalum or other suitable material so that it serves both as a brace and as a radiopaque marker element.

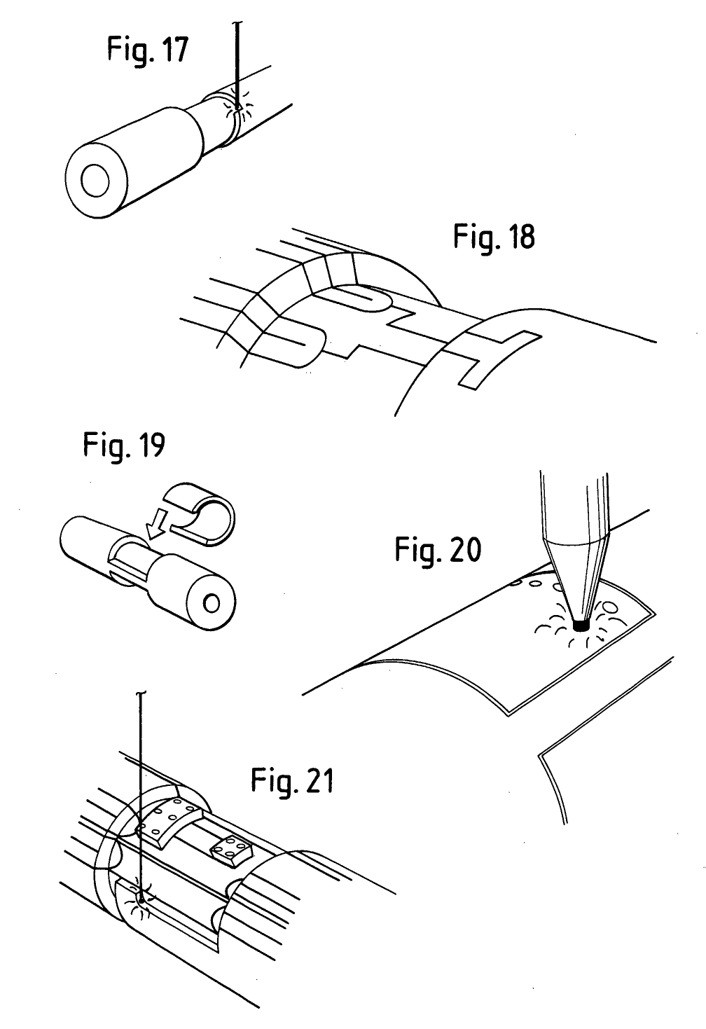

One way to manufacture such a joint with a brace is illustrated in FIGS. 17-21. In FIG. 17 a laser is used to create a recess in a tubular workpiece of nickel titanium alloy. In FIG. 18 first paths for the stent joint is cut. In FIGS. 19-20, a “c” clip of titanium is attached and spot-welded into place. FIG. 21 illustrates how in a second laser operation the unwanted sections of the “c” clip are removed to leave the clip unencumbered with surplus material.

As used herein, the term “joint surface,” as understood by those skilled in the art, is a surface that can cooperate with another surface to constrain movement of the surface and another surface in at least one direction of movement with respect to the longitudinal axis of the medical implant device.

Although the exemplary embodiments described and claimed herein are provided with a variety of particular features to allow a medical implant (e.g., framework, filter, stents or stent-graft) to achieve suitable flexibility with sufficient radial force, variations of the features of the various joints or specific medical implants are permitted to allow the medical implants to be utilized in a mammalian body. These variations may include joint surfaces that are not only flat planar surfaces but are curved planar surfaces; H-shaped center joint part which can be X-shaped while maintaining its ability to retain one more joint member; joint surfaces formed by more than two cut lines (e.g., lines 40/42 and 50/52), three cut lines (e.g., FIGS. 7, 8, and 16B), or multiple cut lines or by undercutting a joint member instead of welding a brace 302 as described in the embodiment of FIG. 16A; or the tie-element 110 between each joint member (e.g., FIG. 11) being replaced with one or more of the joints described herein; the dimensions of the various members such as for example, the struts 14, 16, 18, 20 being from about 15 microns to about 300 microns in width and thickness of about 50 microns to about 300 microns and in some cases, substantially thicker or thinner; the dimensions of various components of the joint being in the same range of about 15 microns to about 300 microns with the requirement that the joint being able extend in at least one direction (e.g., axially parallel to the longitudinal axis of the framework, axially inclined with respect thereto, or curvilinearly thereto) of about 0.1 millimeters to about 4 millimeters or more.

Furthermore, where the joint is employed as part of a stent, such stent would be utilized in various applications, one of which is in the femoral artery. Regardless of the particular application of the stents, the stents can be interconnected by the exemplary joints described herein or a combination of solid connectors and joints. Where the joint is employed as part of a stent-graft, a volume of free space can be provided in the graft covering (e.g., ePTFE or polyurethane) between the stent frameworks to allow for movements of the joint as the stent-graft is flexed.

While the present invention has been disclosed with reference to certain preferred embodiments, numerous modifications, alterations, and changes to the described embodiments are possible without departing from the sphere and scope of the present invention, as defined in the appended claims. Accordingly, it is intended that the present invention not be limited to the described embodiments, but that it have the full scope defined by the language of the following claims, and equivalents thereof.

1. A medical implant for a bodily lumen, the implant having an annular wall between a luminal surface and an abluminal surface, a longitudinal axis, and first and second structural portions, each of the two portions has at least one joint surface extending through the annular wall, with the respective joint surfaces of the first and second portions facing each other to define a joint interposed between the first and second portions that permits movement of the joint surfaces, relative to each other.

2. The implant as claimed in claim 1 wherein the first and second portions are able to move relative to each other in a direction perpendicular to the longitudinal axis of the implant.

3. The implant as claimed in claim 1, wherein the first and second portions are able to move in a direction parallel the longitudinal axis of the implant.

4. The implant as claimed in claim 1, wherein at lease one joint is one of a plurality of joints distributed at spaced intervals around the circumference of the implant.

5. The implant as claimed in claim 1, wherein the implant is radially compressible, to a transluminal delivery disposition.

6. The implant as claimed in claim 1, wherein the implant is a stent.

7. The implant as claimed in claim 1, wherein the joint surfaces are obtainable by cutting with a jet cutter.

8. The implant as claimed in claim 1, wherein the joint surfaces are so constructed and arranged that the first and second portions may come from a single workpiece out of which the joint is formed.

9. The implant as claimed in claim 1, wherein the joint surfaces are so constructed and arranged that they interlock mechanically, in order that the first and second portions are not separable at the joint in the absence of deformation of the joint surfaces.

10. The implant as claimed in claim 1, wherein the joint surfaces of one of the portions become stressed when the joint is in an extended state in order to actively grip the joint surfaces of the other of the two portion, in the extended state of the joint.

11. The implant as claimed in claim 1, wherein the joint surfaces have at least one portion that does not project through a longitudinal central axis of the implant.

12. The implant as claimed in claim 1, and including a brace or bride fixed to one of the first and second portions for inhibiting separation of the first and second portions.

13. The implant as claimed in claim 1, wherein the at least one joint comprises a plurality of joints, and each joint of the plurality of joints has a joint center element, and a tie element which attaches to each joint center element to an adjacent center joint elements, to form a ring of connected element around the longitudinal axis of the implant.

14. A method of making a medical implant for a bodily lumen, the implant having an annular wall between a luminal surface and an abluminal surface, and including first and second structural portions, comprising:
forming in each of the two portions at least one joint surface extending through the annular wall, with the respective joint surfaces of the first and second portions facing each other to define a joint interposed between the first and second portions, that permits movement of the joint surfaces relative to each other.

15. The method as claimed in claim 14, further comprises arranging the joint surfaces to interlock, so the first and second portions are not separable at the joint without deforming the joint surfaces.

16. The method as claimed in claim 14, further comprises creating the joint surfaces by cutting a workpiece with a jet cutter.

17. The method as claimed in claim 16, wherein the jet cutter is a laser cutter.

18. The method as claimed in claim 14, further comprises providing the first and second portions within a single workpiece.

19. A medical implant device, comprising:
a plurality of frameworks disposed about a longitudinal axis extending through the frameworks; and
at least one joint having a first member coupled to a first framework and a second member coupled to a second framework, the first member having first and second sliding surfaces, the second member having complementary sliding surfaces thereto, the first sliding surface being constrained against movement in a first direction relative to the second member and the second sliding surface being constrained against relative movement in a second direction different from the first direction.

20. The medical implant device of claim 19, wherein the first and second directions comprise opposite generally transverse directions with respect to the longitudinal axis.

21. The medical implant device of claim 19, wherein the first and second sliding surfaces comprise two tapered surfaces as viewed through a cross-section of the medical implant device where virtual extensions of the two tapered surfaces toward the longitudinal axis converge.

22. The medical implant device of claim 21, wherein one of the first and second sliding surfaces comprises two ramped surfaces as viewed through a cross-section of the medical implant device where virtual extensions of the two tapered surfaces toward the longitudinal axis diverge.

23. A medical implant device comprising:
a plurality of frameworks disposed about a longitudinal axis extending through the frameworks, the frameworks defining an inner perimeter surrounded by an outer perimeter about the longitudinal axis; and
a joint connected to portions of the frameworks, the joint having a first member coupled to a second member via sliding surfaces so that both the first and second members are located between outer and inner perimeters of the medical implant device and neither of one of the first and second members surrounds the other member.

24. The medical implant device of claim 23, wherein the first and second members are contiguous to the inner and outer perimeters.

25. The medical implant device of claim 24, wherein the inner perimeter is contiguous to a circular perimeter defined by an inner diameter of the frameworks about the longitudinal axis and the outer perimeter is contiguous to a circular perimeter defined by an outer diameter of the frameworks about the longitudinal axis.

26. A medical implant device comprising:
a plurality of frameworks disposed about a longitudinal axis extending through the frameworks; and
a joint connected to portions of the frameworks, the joint having a first planer surface that constrains a second planar surface form movements relative to an axis radial to the longitudinal axis and both the first and second surfaces are located at about the same distance from the longitudinal axis.

27. A medical implant device comprising:
a plurality of frameworks disposed about a longitudinal axis extending through the frameworks;
a first joint having a first member connected to one framework, the first member having a first generally planar surface disposed along the longitudinal axis;
a second joint having a second member connected to another framework, the second member having a second generally planar surface disposed along the longitudinal axis; and
an intermediate member having respective complementary surfaces to the first generally planar surface and second generally planar surface so that one of the intermediate, first and second joints move relative to the longitudinal axis.

28. The medical implant according to claim 27 wherein one of intermediate, first and second joints translate.

29. The medical implant according to claim 27, wherein one of intermediate, first and second joints rotates.

30. The medical implant according to claim 27, wherein one of intermediate, first and second joints translates and rotates.

31. A method of deploying a medical implant device having a plurality of frameworks disposed about a longitudinal axis extending through the frameworks having a first outer diameter in a first configuration and a second diameter larger than the first diameter in a second configuration, the method comprising:
sliding a first member coupled to one framework, the first member having a pair of complementary surfaces and a second member coupled to another framework, the second member having another pair of complementary surfaces;
constraining the first and second member from moving in opposite radial directions with respect to the longitudinal axis; and
expanding the frameworks from the first diameter to the second diameter.

32. The method of claim 31, wherein the expanding comprises locating an inflatable balloon inside the frameworks and expanding the balloon against the frameworks.
